(19) United States
(12) Patent Application Publication
(10) Pub. No.: US 2010/0009424 A1
(43) Pub. Date: Jan. 14, 2010

(54) Sonoporation systems and methods

(76) Inventors: Natasha Forde, (US); Kenneth W. Coffey, Tulsa, OK (US); Jeffrey Joseph Vaitekunas, Lakeville, MN (US)

Correspondence Address: JEFFREY J. VAITEKUNAS, 17650 HYDE PARK AVE., LAKEVILLE, MN, 55044, US

(21) Appl. No.: 12/218,300

(22) Filed: Jul. 14, 2008

(51) Int. Cl. C12N 13/00; C12M 1/42

(52) U.S. Cl. 435/173.4; 435/285.2

(57) ABSTRACT

The present invention is directed to devices and methods that apply ultrasonic energy for the purpose of inducing transfection and cell transformation. A sonoporation system in accordance with embodiments of the present invention includes an ultrasonic electrical energy generator connected to an ultrasonic transducer producing stress waves. The ultrasonic transducer is connected to a fluid containment tank configured to accept at least a portion of the ultrasonic transducer whereby the ultrasonic stress waves may be delivered into the fluid medium. A cell holder is configured to hold one or more cells desirable for transfection. A hydrophone may be electrically connected to an acoustic stress wave intensity detection circuit. A motion control system having an arm configured to receive one or both of the cell holder and the hydrophone is configured to provide motion of one or both of the cell holder and the hydrophone within the fluid medium.

### FIELD OF THE INVENTION

The present invention relates, in general, to the application of ultrasonic energy to cells, mixtures, or biological tissues for the purpose of inducing transfection and cell transformation, and more particularly, to methods and systems for applying ultrasonic energy to cells to induce sonoporation.

### BACKGROUND OF THE INVENTION

Gene therapy is based on deceiving cells to produce a foreign DNA's protein. Foreign DNA is placed into a target cell, and the cell expresses the DNA as if it were its own. With appropriate promoters and enhancers, the cellular machinery manufactures the protein that is coded on the foreign DNA. This foreign DNA specifically produces a desired protein that is expected to have therapeutic value in the case of gene therapy. The uptake of foreign DNA by a cell is called transfection. Transfection occurs in two manners: transient and stable. Transient transfection occurs when the foreign DNA is expressed by the cell but is not incorporated into the nuclear DNA of the cell. Because of this lack of incorporation, the DNA is generally not passed to the daughter cells upon cell division. Stable transfection occurs when the foreign DNA is incorporated into the nuclear DNA of the cell and the genetic material is passed on to the daughter cells. Gene therapy can take place in vivo or ex vivo. Generally, ex vivo methods involve harvesting a patient's cells, culturing them, transfecting the cells, and re-implanting the genetically altered cells in the patient's body.

Cells are the basic structural and functional units of all living organisms. All cells contain cytoplasm surrounded by a plasma membrane. Most bacterial and plant cells are enclosed in a rigid or semi-rigid cell wall. The cells contain DNA that may be arranged in a nuclear membrane or free in cells lacking a nucleus. While the cell membrane is known to contain naturally occurring channels, compounds that are therapeutically advantageous to cells are typically too large to pass through the naturally occurring channels. Conventional interventional methods for delivering compounds to cells have proven difficult in view of the need for the compounds to pass through the cell membrane, cell wall and nuclear membrane.

Many different techniques attempt to place foreign DNA into a target cell. These techniques can be divided into two broad categories; chemically mediated transfection and physically mediated transfection. Among the chemical techniques are calcium phosphate, viral encapsulation, and lipofection. The major physical forms of transfection are electroporation, particle bombardment, and acoustically mediated transfection designated as sonoporation. Electroporation utilizes electric fields to form small pores in the membrane of a cell allowing for the diffusion of DNA into the cell. The particle bombardment method uses high speed projectiles coated with DNA to mechanically introduce the coated DNA into the cells. Acoustically induced transfection utilizes high energy ultrasound to disrupt the integrity of the membrane of cells leading to the formation of transient pores in the membrane. It also causing the opening of stress activated channels allowing for the uptake of DNA through diffusion. In addition to causing the formation of pores, the force of the ultrasound can also drive the genetic material through the pores in the membrane.

Sonoporation may be enhanced using manufactured gas-filled microbubbles. It is known that microbubbles oscillate, or stably cavitate, in an ultrasound field and induce the formation of transient cell membrane pores (sonoporation) through which diffusion of macromolecules can occur. The gene delivery process may, additionally or alternatively, involve inertial cavitation. Inertial cavitation is the violent destruction of the microbubble in a high pressure ultrasound field, which may result in release of the DNA from the shell, sonoporation and perhaps the propulsion of DNA-coated shell fragments into surrounding cell membranes.

For reasons stated above, and for other reasons which will become apparent to those skilled in the art upon reading the present specification, there is a need for systems and methods that provide for improved DNA uptake in cells, designated transduction efficiency, and reduced cell mortality resulting from the transfection process. There is a particular need for improved sonoporation systems and methods that increase transduction efficiency and reduce cell mortality. The present invention fulfills these and other needs, and addresses deficiencies in known systems and techniques.

### SUMMARY OF THE INVENTION

The present invention is directed to devices and methods that apply ultrasonic energy to cells, mixtures, or biological tissues for the purpose of inducing transfection and cell transformation, and to methods and systems for applying ultrasonic energy to cells to induce sonoporation.

One example of a sonoporation system in accordance with the present invention includes a generator configured to provide electrical energy at an ultrasonic frequency. The generator is electrically connected to an ultrasonic transducer configured to convert the electrical energy to ultrasonic stress waves. The ultrasonic transducer is connected to a fluid containment tank having a bottom and at least one side, the at least one side extending from the bottom of the fluid containment tank to a top. The fluid containment tank is configured to contain a fluid medium and further configured to accept at least a portion of the ultrasonic transducer, thereby acoustically coupling the ultrasonic transducer with the fluid medium, whereby the ultrasonic stress waves may be delivered into the fluid medium from the ultrasonic transducer. A cell holder is configured to hold one or more cells desirable for transfection. A hydrophone may be electrically connected to an acoustic stress wave intensity detection circuit. A motion control system is connected to the fluid containment tank, the motion control system having an arm configured to receive one or both of the cell holder and the hydrophone, the arm configured to provide motion of one or both of the cell holder and the hydrophone within the fluid medium.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 illustrates a system for application of ultrasonic waves in accordance with embodiments of the present invention;

FIG. 2 is a side view of a fluid containment tank and components useful for sonoporation in accordance with embodiments of the present invention;

FIG. 3 illustrates a sound field useful in a sonoporation system in accordance with embodiments of the present invention; and

FIG. 4 is a flow chart of a method in accordance with embodiments of the present invention.

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

### DETAILED DESCRIPTION OF THE INVENTION

The present invention relates, in general, to the application of energy to cells, mixtures, or biological tissues for the purpose of inducing transfection and cell transformation, and more particularly, to methods and systems for applying ultrasonic energy to cells to induce sonoporation.

Methods and devices employing sonoporation systems and methods in accordance with the present invention may incorporate one or more of the features, structures, methods, or combinations thereof described herein below. For example, sonoporation systems and methods may be implemented to include one or more of the features and/or processes described below. It is intended that such a device or method need not include all of the features and functions described herein, but may be implemented to include one or more features and functions that, alone or in combination, provide for unique structures and/or functionality.

The present invention is directed to devices and methods that apply ultrasonic energy for the purpose of inducing transfection and cell transformation. A sonoporation system in accordance with embodiments of the present invention includes an ultrasonic electrical energy generator connected to an ultrasonic transducer producing stress waves. The ultrasonic transducer is connected to a fluid containment tank configured to accept at least a portion of the ultrasonic transducer whereby the ultrasonic stress waves may be delivered into the fluid medium. A cell holder is configured to hold one or more cells desirable for transfection. A hydrophone may be electrically connected to an acoustic stress wave intensity detection circuit. A motion control system having an arm configured to receive one or both of the cell holder and the hydrophone is configured to provide motion of one or both of the cell holder and the hydrophone within the fluid medium.

Acoustically induced transfection is based on cavitation. Cavitation refers to the formation of microbubbles of gas in a high-intensity acoustic field. Because of its relatively high frequency, ultrasound is typically transmitted through a liquid medium so that it does not dissipate. Dissolved gas in this liquid medium tends to come out of solution during the low pressure stage of the acoustic wave. During the high pressure portion of the compression wave, the gas attempts to dissolve back into the solution, but because of differences in the surface area of the bubble, the bubble gains more gas during the low pressure period than it loses during the high pressure period.

With each cycle of the ultrasound wave, the bubble gains gas until it reaches equilibrium and the gases entering it are equal to the gases escaping or, alternatively, it reaches resonant diameter. If it reaches resonant diameter, the bubble is torn apart and the energy of the acoustical field is concentrated, in certain conditions it may increase 10 orders of magnitude or more. This high concentration of power may cause the formation of transient pores in the membrane of nearby cells and allow for the passive uptake of plasmid DNA.

In applications where manufactured microbubbles are used, the concentration of the microbubble nuclei in the mixture is typically between 6E+6 bubbles/ml and 300E+6 bubbles/ml. The concentration of bubble micronuclei is typically at least 10% by volume. The frequency of the ultrasonic waves is preferably in the range of about 0.1 to about 3.0 MHz. Typically, the intensity of the ultrasonic waves is in the range of 0.1-10 Watts/cm².

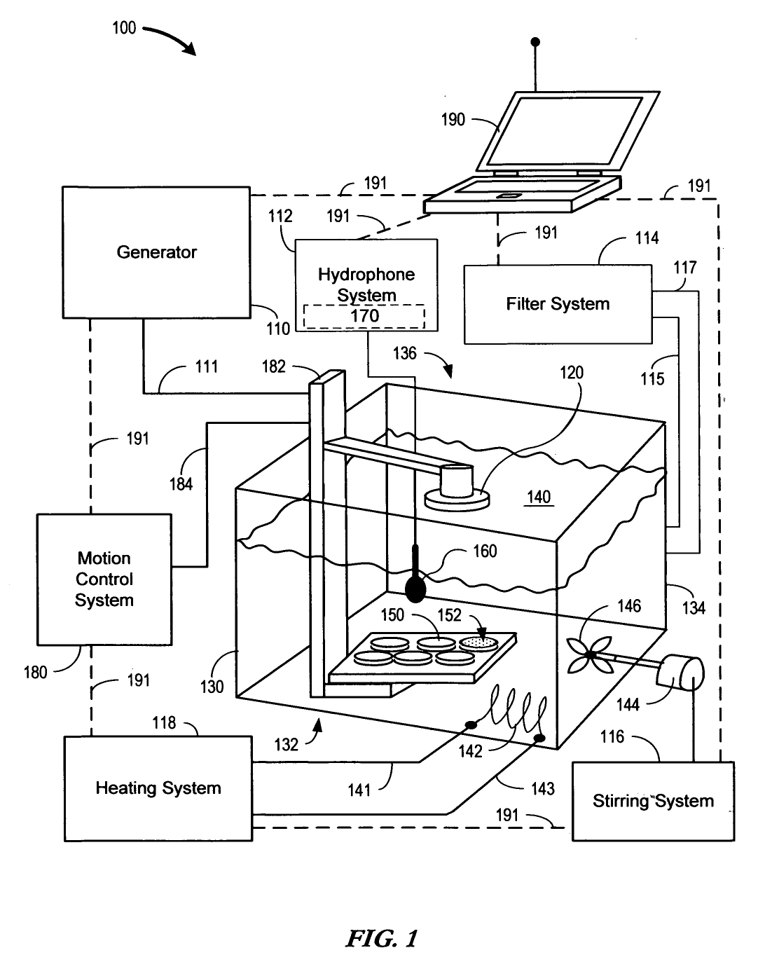

FIG. 1 illustrates a system 100 for application of ultrasonic waves in accordance with embodiments of the present invention. System 100 includes a generator 110 configured to provide electrical energy at an ultrasonic frequency. The generator 110 is electrically connected to an ultrasonic transducer 120 configured to convert the electrical energy to ultrasonic stress waves. The ultrasonic transducer 120 is combined with a fluid containment tank 130 having a bottom 132 and at least one side 134, the at least one side 134 extending from the bottom of the fluid containment tank 130 to a top 136. The fluid containment tank 130 is configured to contain a fluid medium 140 and further configured to accept at least a portion of the ultrasonic transducer 120, thereby acoustically coupling the ultrasonic transducer 120 with the fluid medium 140, whereby the ultrasonic stress waves may be delivered into the fluid medium 140 from the ultrasonic transducer 120. A cell holder 150 is configured to hold one or more cells 152 desirable for transfection. For example, suitable cell holders include an OPTICELL cell holder, trademark of Thermo Fisher Scientific under the NUNC brand, Pittsburgh Pa.; a SONOPORE cell holder, a trademark of Nepa gene in Japan; a SONIDEL cell holder, a trademark of SONIDEL Limited, Ireland, contained in the SONIDEL STK10 kit,; or other suitable cell plate (Nunc Multidishes Nunclon™ tissue culture well plates, 6, 12, 24, 48 well plates), cell holder, or cell containment system.

A hydrophone 160 may be electrically connected to an acoustic stress wave intensity detection circuit 170. A motion control system 180 is connected to the fluid containment tank 130, the motion control system 180 having an arm 182 configured to receive one or both of the cell holder 150 and the hydrophone 160, the arm 182 may be configured to provide motion of one or both of the cell holder 150 and the hydrophone 160 within the fluid medium 140.

Fluids suitable for use in the system 100 include, for example, degassed deionized water, as well as fluids that may be used to mimic tissue. For example, castor oil may be used to provide an acoustic impedance and acoustic attenuation similar to tissues of interest. Other suitable tissue mimicking materials include a scatterer in finely divided form uniformly dispersed throughout a liquid having in the absence of particulate scatterers a speed of sound within the range of 1400-1650 m/s. The liquid may include water and an organic hydroxy compound soluble in the water. A tissue mimicking fluid should be capable of mimicking soft tissue with respect to speed of sound, i.e. from 1460 m/s for fat to 1640 m/s for an eye's lens; with respect to attenuation coefficient, i.e. from 0.4 dB/cm/MHz for fat to 2.0 dB/cm/MHz for muscle; and with respect to scattering coefficients. The attenuation coefficient is approximately proportional to the ultrasonic frequency.

A processor 190, such as a computer, may be used to control subsystems within the system 100. For example, the processor 190 may be connected to one of more of the generator 110, a hydrophone system 112, a filter system 114, a stirring system 116, a heating system 118, and the motion control system 180. The processor may be communicably coupled using a local area network 191 such as Ethernet, direct wiring, a short-range wireless communication interface, such as an interface conforming to a known communications standard, such as Bluetooth or IEEE 802 standards, or other communication method. The system 100 may incorporate some or all of the subsystems into the processor 190 or into a stand-alone chassis, for example.

The stirring system 116 may be electrically connected to a motor 144, for example. The motor 144 may drive a propeller 146 within the fluid medium 140 to stir the fluid medium 140. Alternatively, the stirring system 116 may be positioned external to the fluid containment tank 130 and stir the fluid medium 140 using a magnetic stirring method, for example using a Cimarec 2 magnetic stirring system, model SP46925, available from Barnstead/Thermodyne, Dubuque, Iowa.

The heating system 118 may include a resistive heating element 142 immersed in the fluid medium 140, and electrically coupled to the heating system 118 using a positive lead 141 and a negative lead 143. Alternatively, the heating system 118 may be positioned external to the fluid containment tank 130 and heat the fluid medium 140 using a hot plate under the bottom 132, for example using the Thermodyne hot plate included with the Cimarec 2 magnetic stirring system, model SP46925, available from Barnstead/Thermodyne, Dubuque, Iowa.

The filter system 114 may be used to keep the fluid medium 140 free of bacteria and debris, such as by using a fish tank filter available from pet supply stores. The filter system 114 may include a pump that may be used as an alternative to the motor 144, thereby incorporating the stirring system 116 into the filter system 114. If, for example, the fluid medium 140 is drawn from the fluid containment tank 130 near the bottom 132, via a fluid path 117, the filter system 114 may filter the fluid medium 140, and return it to the fluid containment tank 130 via a pathway 115 near the top 136. This would set up a continuous flow of the fluid medium 140 from the top 136 of the fluid containment tank 130 to the bottom 132 of the fluid containment tank 130, keeping the fluid medium 140 sufficiently stirred. Further, the heating system 118 may be incorporated into the filter system 114. Therefore, in embodiments of the present invention, the heating system 118, the stirring system 116, and the filter system 114, may be combined into a single subsystem of the system 100. In alternate embodiments, the fluid medium may be pumped past ultraviolet radiation to sterilize the fluid. In still other embodiments the fluid may be pumped through a degassing system to degas the fluid. In further embodiments, a gas sensor such as an oxygen sensor may be used to determine the dissolved gas concentration of the fluid.

The hydrophone system 112 is electrically connected to the hydrophone 160, which may be permanently or removably connected to the fluid containment tank 130. In embodiments of a system 100 in accordance with the present invention, the hydrophone 160 may be configured to removably replace the cell holder 150 on the arm 182 of the motion control system 180. A suitable hydrophone system 112 may be, for example, a LeCroy Digital Oscilloscope, model 9354AM, manufactured by LeCroy in Switzerland. The hydrophone may be connected directly to the oscilloscope, or may be connected using a preamp, such as a Panametrics model 5678 Preamp, made by Panametrics Corp., Waltham, Mass. In another example, the acoustic stress wave intensity detection circuit 170 may be a peak-hold circuit or other suitable circuitry electrically connected to an A/D converter in the processor 190.

The generator 110 may drive the ultrasonic transducer 120 continuously, continuously pulsed, or intermittently, while the hydrophone 160 is scanned within the fluid containment tank 130 using the motion control system 180. The acoustic stress wave intensity detection circuit 170 may be used to detect the intensity of the ultrasonic energy, and intensities as a function of location may be recorded by the processor 190. For example, it may be desirable to know, for a particular ultrasonic transducer 120, what the intensities are within the fluid containment tank 130 to plan a dosimetry for the cells 152 in the cell holder 150. The intensity map may be used to position the cell holder 150 at a particular location within the fluid medium 140, or may be used to plan a trajectory of the cell holder 150 within the fluid containment tank 130. The motion control system 180 may be used to impart a particular trajectory to the cell holder 150 within the fluid medium 140.

The motion control system may be used to position a sample or object, such as an in-vitro tissue sample, relative to the transducer 120. For example, if a tissue mimicking fluid is used as the fluid medium 140, the system 100 may be used to evaluate the effects of the transducer 120 on the in-vitro tissue sample. The system 100 may be used to simulate having the in-vitro tissue sample at different depths using the tissue mimicking fluid.

Alternatively, the cell holder 150 may be held stationary and the ultrasonic transducer 120 may be moved using the motion control system 180. For example, the motion control system 180 may control multiple axes of motion, and the hydrophone 160, the cell holder 150, and the ultrasonic transducer 120 may each be independently moveable within the fluid medium 140. The processor may be used to calculate the far field of the transducer, and the calculated distance may be used to position the cell holder relative to the transducer. In another embodiment, the hydrophone may be used to empirically determine the far field of the transducer, and the determined distance may be used to position the transducer or cell holder.

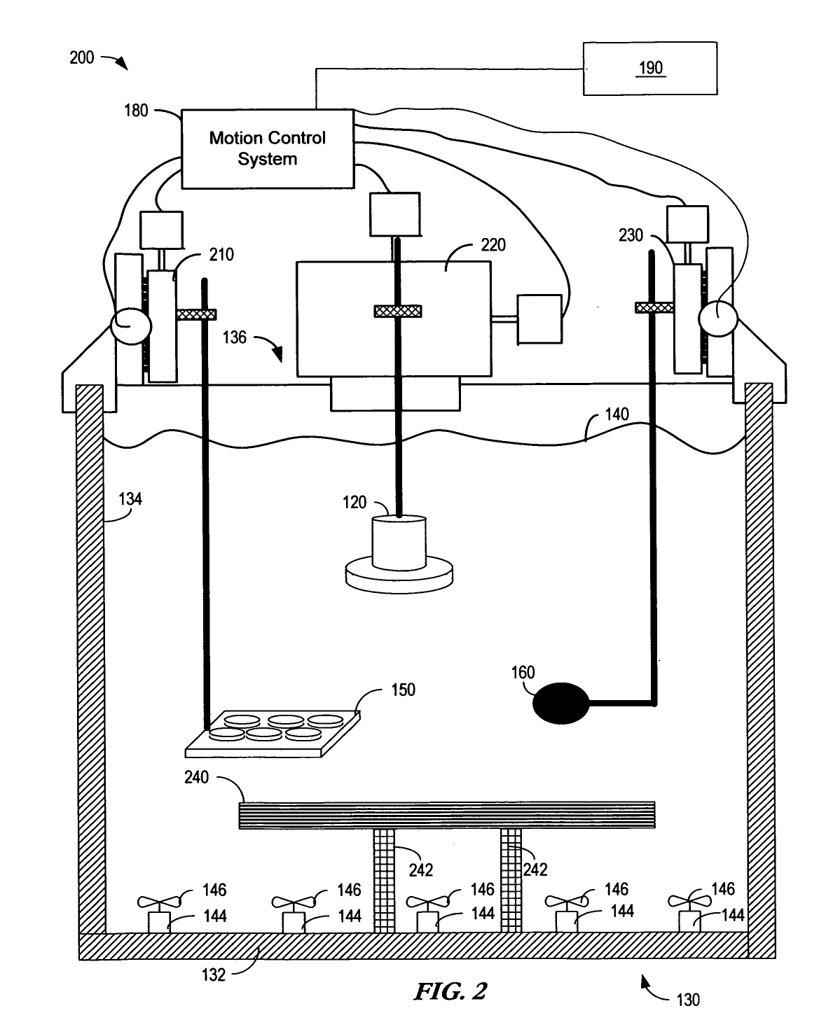

FIG. 2 is a side view of the fluid containment tank 130 and components useful for sonoporation in accordance with embodiments of the present invention. A sonoporation system 200 is illustrated that incorporated the fluid containment tank 130 with the motion control system 180 having multiple axes of motion. Motion stages 210, 220, and 230 are illustrated connected to the cell holder 150, the ultrasonic transducer 120, and the hydrophone 160 respectively. The motion stages 210, 220, 230 are illustrated as having two degrees of freedom (axes of motion) for illustrative clarity, and not as limitation. It is contemplated that any number of degrees of freedom may be used with any number of motion stages. In a particular scenario, the hydrophone 160 and ultrasonic transducer 120 may be moved relative to one another until an appropriate location is determined for the location of the cell holder 150. The location appropriate for cell holder 150 may be recorded by the processor 190.

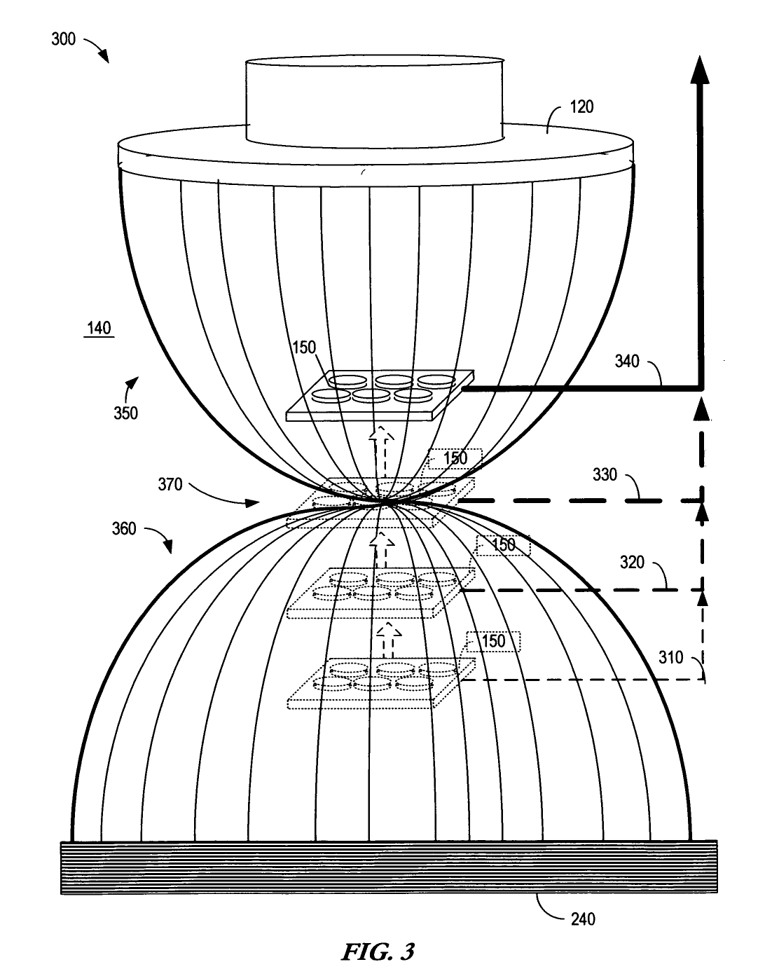

In FIG. 3, the bottom 132 of the fluid containment tank 130 contains five replications of the motor 144 and propeller 146, which may be controlled in unison in one embodiment, or independently or coupled in groups in other embodiments, to prescribe flow of the fluid medium 140. An acoustic reflector 240 is illustrated parallel to the bottom 132 of the fluid containment tank 130, resting on pillars 242. Although the acoustic reflector 240 is illustrated as stationary and parallel to the bottom 132, it is contemplated that the acoustic reflector 240 may be positionable by the motion control system 180 or manually moveable and adjustable for height and/or angle. The acoustic reflector 240 may be, for example, a hollow shell containing air, or a polymeric material containing air-filled spheres, or a closed-cell foam containing air or other gas, or other material having a significantly different acoustic impedance from the fluid medium 140, such that the acoustic reflector 240 reflects a significant portion of acoustic energy. By reflecting acoustic energy propagating from the ultrasonic transducer 120, for example, a sound field may be developed in the fluid medium 140 that is particularly suitable for sonoporation.

Referring to FIG. 3, a representative sound field of the ultrasonic transducer is illustrated in accordance with embodiments of the present invention. The motion control system 180 may be used to move the hydrophone 160 to a location suitable for measuring a dose at the recorded location. For example, a relationship may be determined between the intensity of the ultrasonic energy at the recorded location and a hydrophone measurement location. The relationship may be used, such as by using a look-up table or algorithmic calculation, to determine the dosage at the cell holder 150 location using the hydrophone 160 measurement at the hydrophone measurement location. For example, an equation for the far-field intensity may be used to determine a suitable location within the interference zone or beyond the interference zone in the far field for cell holder placement. Suitable equations for field equations may be found, for example, in Kinsler and Frey's Fundamentals of Acoustics, ISBN 0-471-02933-5.

In FIG. 3 the ultrasonic transducer 120 is illustrated propagating an ultrasonic stress wave toward the acoustic reflector 240, which is then reflected back towards the ultrasonic transducer 120, in this case setting up a standing wave illustrated by intensity lines 350, 360 and having a node location 370. The intensity lines 350, 360 illustrate that the acoustic intensity of the standing wave is highest at the node 370, and that the intensity decreases as the distance from the node 370 to the ultrasonic transducer 120 increases, illustrated by intensity lines 350, or as the distance from the node 370 to the acoustic reflector 240 increases, illustrated as intensity lines 360.

In one embodiment of a method of transfecting cells illustrated in FIG. 3, the cell holder 150 is illustrated as moving from a first location 310, through a second location 320. The cell holder 150 continues through a third location 330 that coincides with the node 370, and is illustrated at current position 340 in the ultrasound field illustrated by intensity lines 350. The cell holder 150 may be moved continuously or in coordination with acoustic intensity modulation, varying pulse parameters, or coordinated with other events such as heating cycle, stirring parameters, hydrophone measurements or other desirable combinations.

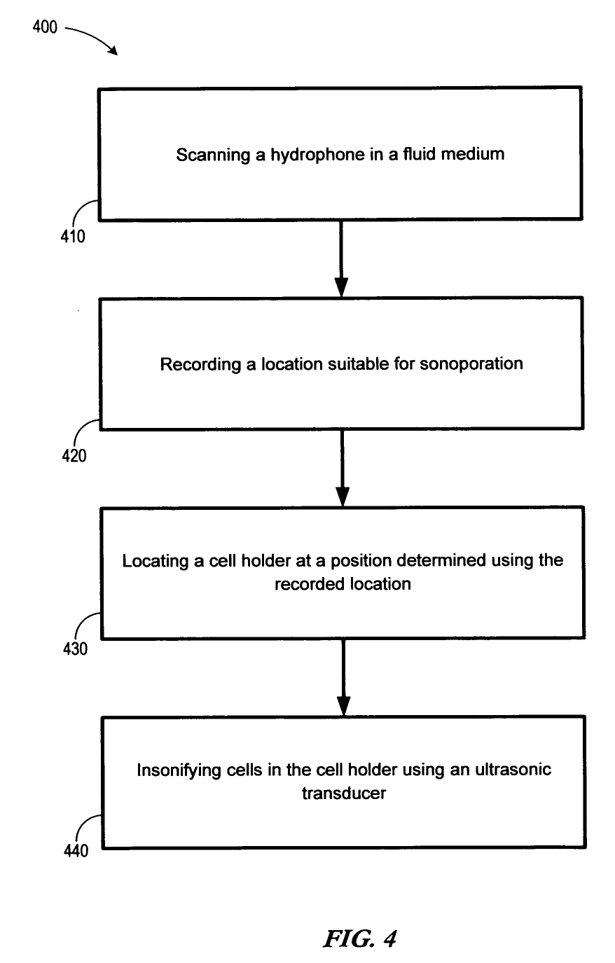

Referring to FIGS. 1-3, although a single node 370 is illustrated in FIG. 4 for purposes of clarity, and not limitation, any number of nodes may be set up in the fluid medium 140. For example, multiple nodes may be arranged to form a horizontal array of nodes, a vertical array of nodes, or a three-dimensional array of nodes relative to the top 136 and the bottom 132 of the fluid containment tank 130. The cell holder 150 may, for example, be programmed to move through an array of nodes such that the cells 152 experience a defined repetitive acoustic intensity modulation.

In another embodiment in accordance with the present invention, two or more transducers 120 may be used to create an array of standing wave patterns that are configured to coincide with wells of the sell holder 150 in order to insonify a number of wells containing a number of the same or different cells 152 simultaneously as the cell holder 150 is moved through the sound field.

In further embodiments, the cell holder 150 may be held stationary, and two or more frequencies and/or two or more transducers may be used to vary the sound field in the fluid medium 140. For example, two frequencies may be simultaneously propagated from the transducer 120 to set up a traveling beat acoustic pattern in the fluid medium 140. The acoustic intensity will then beat against the cells 152 in the cell holder 150 as the acoustic field propagates past the cell holder 150. Alternatively, the cell holder may move in coordination with the beating of the multiple frequencies. In another embodiment, the relative difference between the two simultaneous frequencies may be modulated to vary the beat frequency. In still another embodiment, the relative difference between the two simultaneous frequencies may be modulated sinusoidally, such that the beating pattern moves in a reciprocating motion through the cells 152 such that during one full beat cycle the intensity varies from above the cells 152 to below the cells 152, and then from below the cells 152 back above the cells 152.

The node or beat pattern of the acoustic intensity field may be used to drive cavitation bubbles and/or microbubbles relative to the cells 152. For example, in a standing wave field, bubbles and/or microbubbles will be driven towards the node. If the cells 152 are positioned towards the node of the standing wave relative to the bubbles/microbubbles, then the bubbles/microbubbles will be driven towards the cells 152. Driving the bubbles/microbubbles toward or away from the cells before moving the cells through the node may provide improved transfection rates. As described above, movement of the cells 152 with respect to the node may be accomplished in several ways in addition to physical positional movement. Suitable microbubbles include, for example, ARTISON microbubbles available from ARTISON Corp., Inola, Okla.

FIG. 4 is a flow chart of a method 400 in accordance with embodiments of the present invention. Method 400 includes scanning a hydrophone in a fluid medium, designated as step 410, recording a location suitable for sonoporation, designated as step 420, locating a cell holder at a position determined using the recorded location, designated as step 430, and insonifying cells in the cell holder using an ultrasonic transducer, designated as step 440.

Referring now to FIGS. 1 through 4, ultrasound output intensity of a particular transducer may be measured by, for example, scanning a calibrated 0.5 mm diameter hydrophone (such as a NTR Model NP-1000, Seattle, Wash.) 4 mm in front of the transducer face in a tank of de-gassed water or tissue mimicking fluid or other desirable fluid. Using this methodology, the intensities are typically described as spatial average temporal peak (SATP) in the freely propagating near field of the transducer. The actual exposure conditions may be standing wave conditions if the transducer is located below the sample and directed up to the surface of the water, because the sound reflects from the free surface of the water. Thus, the pressures within the media may reach twice the free field values resulting in intensities as high as four times the SATP measured values in standing wave conditions.

The ultrasound may be applied in continuous wave or in pulsed mode. The pulsing may be swept along a frequency range, e.g., an increasing swept-frequency pulse (CHIRP) or frequency-decreasing CHIRP (PRICH) pulses may be employed. This may be accomplished by using a broadband transducer and sweeping the transducer through the frequency range of the bandwidth of the transducer with a pulse function generator. For pulsed applications, pulse repetition frequency may range between about 0.01 Hz and about 10 kHz, and typically between about 0.03 Hz and about 1 kHz. The pulse may incorporate a duty cycle between about 0.1 and about 99.9%, and typically is between about 5% and about 50%.

Further, more than one frequency of sound may be employed, e.g., 20 kHz with 1 MHz or 250 and 500 kHz with 1 MHz as non-limiting non-exhaustive examples. When more than one frequency of sound is applied to the cells, the pulsing of the different frequency components may be modulated such that additive effects are optimized through interaction of the different frequency components. For example, the time duration between application of one or more pulses of 20 kHz and subsequent application of 1 MHz may be adjusted so that the higher frequency waves arrive at a time t, where t=λ/4 C at 20 kHz, where λ is the acoustic wavelength in the medium and C is the speed of propagation of sound in the fluid.

The ultrasound intensity may be applied between a range of about 1 microwatt/cm² and about 100 watts/cm², and is typically within the range between about 0.1 and about 5 watts/cm². The ultrasound energy's peak pressure may vary between about 1 Pascal and about 1,000 MegaPascals, and is typically in the range between about 50 kilopascals and 50 MegaPascals.

Typical retroviral particles useful in accordance with the present invention include murine leukaemia virus (MLV), human immunodeficiency virus (HIV) or equine infectious anaemia (EIAV), for example. A retroviral particle may be recombinant. A retroviral particle may be associated with a manufactured microbubble. The retroviral particle may be associated with the microbubble by, for example, electrostatic interaction, affinity cross-linking or covalent attachment.

The shell of a manufactured microbubble typically comprises lipid. The shell of a microbubble may be manufactured from, for example, albumin or methacrylate. The shell of the microbubble may be cationic. The gas in a microbubble may be, for example, perfluorocarbon, sulphur hexafluoride, air, or other suitable gas.

A microbubble or retroviral particle, or composition may further comprise a targeting moiety. The targeting moiety may be, for example, a ligand or an antibody. Where the targeting moiety is an antibody, the antibody may be, for example, an anti-ICAM-1 antibody, anti-E-selectin antibody, anti-VEGF receptor antibody or anti-αᵥβ₃ antibody.

A gene of interest is typically a therapeutic gene. A gene of interest may be one which is not functionally expressed in a target cell. A gene of interest may be one for which lack of functional expression in an individual is causative of disease in that individual. Lack of functional expression in an individual may be due to the presence of a mutated form of the gene, absence of the gene, or lack of functional gene product, in the individual. The gene of interest may not be functionally expressed in a target cell where it is not naturally expressed in the target cell. For example, the gene of interest may be adenosine deaminase, VEGF, GM-CSF, factor VIII, factor IX, CFTR, p53, TNFα, TIMP-3 or thymidine kinase.

Manufactured microbubbles suitable for use with ultrasound may be used in accordance with the present invention, including, for example, microbubbles having a shell manufactured from a lipid, albumin or methacrylate. Examples of commercially available microbubbles are ARTISON, DEFINITY and SONOVUE having a lipid shell, and OPTISON having a shell comprising albumin.

The following describes a method in accordance with embodiments of the present invention for isolating and transfecting mammalian cells. The method may be readily adaptable to other cell types. In designing a method for a particular cell type, one would examine various parameters. For example volume of media, exposure intensities, length of exposure, duty cycles, DNA concentration,and microbubble concentration.

For experimentation the cells were grown in the following manner: The African green monkey kidney fibroblast cell line, COS-7, was cultured in Dulbecco's modified Eagle's medium (DMEM; Sigma Chemical Co., St. Louis, Mo.) supplemented with 10% heat-inactivated fetal bovine serum (FBS, GIBCO, Invitrogen Co., Carlsbad, Calif.). Cells are passaged 24 hours prior to sonoporation as per specific cell line instructions. Cells are plated in the vessel they will be treated in, i.e. either a 12, 24, 48 tissue culture well plate, or in the specific cell holder. Cells should be in the exponential growth phase for transfection, and so are usually used when they reach 60-70% confluency.

Cell preparation for experiment: Prior to sonoporation, cells are removed from incubator, medium from the cells is removed, cells are washed twice with fresh medium to remove any cell debris and dead cells and fresh medium is then added to the cells. Cells are treated attached to tissue culture plates or in suspension in tubes at app 1×10⁵ cells in 2 mls of growth medium.

The mixture of microbubbles and genetic material to be delivered is prepared. For example, plasmid DNA to be delivered is amplified in a host strain bacteria, it is purified and re-suspended in either PBS or sterile water. Various reporter genes such a luciferase, β-galactosidase and green fluorescent protein are frequently used for determining effective gene delivery. In one preferred embodiment, plasmids, such as pIRES-2-EGFP (5300 bp by BD Biosciences Clontech, Mountain View, Calif.) pSV-.beta.-Galactosidase control vector (6821 bp, Promega Corp., Madison, Wis.) are used for transient and stable transfection, respectively. Amplified plasmids are purified from the bacterial cultures using a plasmid prep kit (Qiagen Inc., Chatsworth, Calif.). After purification of DNA the absorbance of the DNA in solution is read at 260 and 280 nm. The ratio of A 260/280 nm should be between 1.8-1.9.

Other molecules which may be delivered in accordance with embodiments of the present invention include macromolecules such as DNA, RNA and protein. However, other molecules, such as vitamins and other therapeutic moieties, are suitable for use in accordance with embodiments of the present invention. Additionally, one may wish to deliver therapeutic substances, such as calcium, by embodiments of methods in accordance with the present invention.

A suitable cocktail solution may include microbubbles, free radical scavengers, DNAse, RNAse and protease inhibitors, and phospholipids. The ARTISON microbubbles are composed of a lipid shell filled with perfluorocarbon gas. For experimentation the bubbles are re-dispersed by gently shaking the vial end-to-end for 10 seconds. The suspension should appear uniformly opaque. The vial is vented with a 25 G needle. The desired volume is with drawn from the vial and then the venting needle is removed and vial is stored in the refrigerator. Bubbles are combined with the DNA. ARTISON bubbles are used at a starting point of 5-10% of the final volume.

The DNA which can be suspended in either sterile water or phosphate buffered saline. The bubbles are added to the tube contained the DNA and the solution is mixed gently for 10 seconds to allow the DNA to combine with the microbubbles. The solution is then added directly to the cells and not allowed sit longer than 30 seconds as the bubbles may come out of solution, e.g. for a 24 well plate add 10-40 ug plasmid+10% ARTISON bubbles+2 mls medium.

The Sonitron probe is placed down into the medium in the well and cells were treated using the following conditions:
⅓ MHz
0.5-2 W/cm2
10-100% Duty cycle
10-60 sec

Following sonoporation cells are washed twice with PBS then returned to incubator and left for 24, 48 72 hours to examine gene expression. Depending on gene of interest uptake can be quantified using flow cytometry or fluorescent microscopy, luciferase assay or β-galactosidase staining and microscopy.

It is understood that the components and functionality depicted in the figures and described herein may be implemented in hardware, software, or a combination of hardware and software. It is further understood that the components and functionality depicted as separate or discrete blocks/elements in the figures may be implemented in combination with other components and functionality, and that the depiction of such components and functionality in individual or integral form is for purposes of clarity of explanation, and not of limitation.

Illustrations of method steps, such as, for example, the steps illustrated in FIG. 4, show steps sequentially and in a particular order. There is no need to perform the steps in the order illustrated. Deviating from the illustrated order for some or all of the steps is contemplated by the inventor, and does not depart from the scope of the present invention.

Each feature disclosed in this specification (including any accompanying claims, abstract, and drawings), may be replaced by alternative features having the same, equivalent or similar purpose, unless expressly stated otherwise. Thus, unless expressly stated otherwise, each feature disclosed is one example only of a generic series of equivalent or similar features.

While preferred embodiments of the present invention have been shown and described herein, it will be obvious to those skilled in the art that such embodiments are provided by way of example only. Numerous variations, changes, and substitutions will be apparent to those skilled in the art without departing from the invention. Accordingly, it is intended that the invention be limited only by the scope of the appended claims.

What is claimed is:

1. A sonoporation system, comprising:
a generator configured to provide electrical energy at an ultrasonic frequency;
an ultrasonic transducer electrically connected to the generator and configured to convert the electrical energy to ultrasonic stress waves;
a fluid containment tank having a bottom and at least one side the at least one side extending from the bottom of the fluid containment tank to a top, the fluid containment tank configured to contain a fluid medium and further configured to accept at least a portion of the ultrasonic transducer, thereby acoustically coupling the ultrasonic transducer with the fluid medium, whereby the ultrasonic stress waves are delivered into the fluid medium from the ultrasonic transducer;
a cell holder configured to hold one or more cells desirable for transfection;
a motion control system connected to the fluid containment tank, the motion control system having an arm configured to receive the cell holder, the arm configured to provide motion of the cell holder within the fluid medium;
a hydrophone contained within the fluid medium, the hydrophone electrically connected to an acoustic stress wave intensity detection circuit;
a stirring system configured to stir the fluid medium within the fluid containment tank; and
a heating system configured to maintain the fluid medium at about a predetermined temperature.

2. The system of claim 1, wherein the fluid containment tank is configured to accept the portion of the ultrasonic transducer through the bottom of the fluid containment tank.

3. The system of claim 1, wherein the arm of the motion control system is configured to receive the cell holder and the hydrophone interchangeably.

4. The system of claim 1, wherein the fluid containment tank further comprises an ultrasonic stress wave reflector configured to reflect the ultrasonic stress waves delivered from the ultrasonic transducer and produce a standing wave in the fluid medium.

5. The system of claim 1, wherein the fluid containment tank further comprises an ultrasonic stress wave reflector configured to reflect the ultrasonic stress waves delivered from the ultrasonic transducer and produce a desired standing wave in the fluid medium and wherein the fluid containment tank further comprises acoustic scattering elements configured to reduce undesired standing waves in the fluid medium.

6. The system of claim 1, wherein the motion control system is configured to vary the distance between the cell holder and the ultrasonic transducer.

7. The system of claim 1, wherein the motion control system is configured to vary the distance between the cell holder and the ultrasonic transducer during delivery of the ultrasonic stress waves from the ultrasonic transducer into the fluid medium.

8. The system of claim 1, wherein the fluid containment tank further comprises an ultrasonic stress wave reflector configured to reflect the ultrasonic stress waves delivered from the ultrasonic transducer and produce a desired standing wave in the fluid medium and wherein the motion control system is configured to vary the distance between the cell holder and the ultrasonic transducer during delivery of the ultrasonic stress waves from the ultrasonic transducer into the fluid medium.

9. A sonoporation system, comprising:
a generator configured to provide electrical energy at an ultrasonic frequency;
an ultrasonic transducer electrically connected to the generator and configured to convert the electrical energy to ultrasonic stress waves;
a fluid containment tank having a bottom and at least one side, the at least one side extending from the bottom of the fluid containment tank to a top, the fluid containment tank configured to contain a fluid medium and further configured to accept at least a portion of the ultrasonic transducer, thereby acoustically coupling the ultrasonic transducer with the fluid medium, whereby the ultrasonic stress waves are delivered into the fluid medium from the ultrasonic transducer;
a cell holder configured to hold one or more cells desirable for transfection;
a hydrophone electrically connected to an acoustic stress wave intensity detection circuit; and
a motion control system connected to the fluid containment tank, the motion control system having an arm configured to receive one or both of the cell holder and the hydrophone, the arm configured to provide motion of one or both of the cell holder and the hydrophone within the fluid medium.

10. The system of claim 9, wherein the sonoporation system further comprises a processor configured to determine the intensity of the ultrasonic stress waves in the fluid medium using the acoustic stress wave intensity detection circuit as the motion control system moves the hydrophone through the fluid medium using the motion control system.

11. The system of claim 9, wherein the sonoporation system further comprises a processor configured to determine the intensity of the ultrasonic stress waves in the fluid medium using the acoustic stress wave intensity detection circuit as the ultrasonic transducer is delivering the ultrasonic stress waves to the cell holder.

12. The system of claim 11, wherein the processor is electrically connected to the generator and the sonoporation system is configured to modulate the amplitude of the electrical energy in response to the determined intensity.

13. The system of claim 11, wherein the processor is electrically connected to the generator and the generator turns off the electrical energy in response to determined intensity exceeding a predetermined value.

14. The system of claim 9, wherein the sonoporation system further comprises a processor configured to determine the intensity of at least a portion of the ultrasonic stress waves in the fluid medium using the acoustic stress wave intensity detection circuit as the ultrasonic transducer is delivering the ultrasonic stress waves to the cell holder, and as the motion control system moves the cell holder through the fluid medium using the motion control system.

15. The system of claim 9, wherein the sonoporation system further comprises a processor configured to determine the intensity of at least a portion of the ultrasonic stress waves in the fluid medium using the acoustic stress wave intensity detection circuit as the ultrasonic transducer is delivering the ultrasonic stress waves to the cell holder, and as the motion control system moves the cell holder through the fluid medium using the motion control system, wherein the generator is configured to receive a signal from the processor, and use the signal to control the electrical energy.

16. The system of claim 15, wherein the generator modulates the amplitude of the electrical energy in response to the signal.

17. The system of claim 15, wherein the generator turns off the electrical energy in response to the signal.

18. The system of claim 15, wherein the fluid is adapted to approximate the acoustic impedance of tissue.

19. The system of claim 15, wherein the fluid is adapted to approximate the acoustic impedance of tissue, and wherein the motion control system is configured to move the cell holder to a position in the tank approximating a predetermined target depth.

20. A sonoporation method, comprising:
providing an ultrasonic transducer;
delivering ultrasonic energy from the transducer into a fluid medium;
scanning a hydrophone in the fluid medium using a motion control system;
determining a location suitable for sonoporation in the fluid medium using the hydrophone;
recording the location from the motion control system, the location;
locating a cell holder at a position determined using the recorded location; and
insonifying cells in the cell holder using the ultrasonic transducer.

21. A sonoporation system, comprising:
a generator configured to provide electrical energy at an ultrasonic frequency;
an ultrasonic transducer electrically connected to the generator and configured to convert the electrical energy to ultrasonic stress waves;
a fluid containment tank having a bottom and at least one side, the at least one side extending from the bottom of the fluid containment tank to a top, the fluid containment tank configured to contain a fluid medium and further configured to accept at least a portion of the ultrasonic transducer, thereby acoustically coupling the ultrasonic transducer with the fluid medium, whereby the ultrasonic stress waves are delivered into the fluid medium from the ultrasonic transducer;
a cell holder configured to hold one or more cells desirable for transfection;
a hydrophone electrically connected to an acoustic stress wave intensity detection circuit;
a motion control system connected to the fluid containment tank, the motion control system having an arm configured to receive one or both of the cell holder and the hydrophone, the arm configured to provide motion of one or both of the cell holder and the hydrophone within the fluid medium; and
a processor configured to determine the intensity of at least a portion of the ultrasonic stress waves in the fluid medium using the acoustic stress wave intensity detection circuit as the ultrasonic transducer is delivering the ultrasonic stress waves, and as the motion control system moves the hydrophone through the fluid medium using the motion control system, wherein the processor is configured to record the intensity, and use the recorded intensity to determine a position suitable for location of the cell holder within the fluid medium.
